(19) United States
(12) Patent Application Publication
(10) Pub. No.: US 2005/0014995 A1
(43) Pub. Date: Jan. 20, 2005

(54) Direct, real-time imaging guidance of cardiac catheterization

(76) Inventors: David Amundson, Boulder, CO (US); H. John Hanlin, Louisville, CO (US); Larry Blankenship, Boulder, CO (US)

Correspondence Address: PETER F WEINBERG;GIBSON DUNN AND CRUTCHER LLP, SUITE 4100, 1801 CALIFORNIA STREET, DENVER, CO, 80202, US

(21) Appl. No.: 10/495,037

(22) PCT Filed: Nov. 12, 2002
(86) PCT No.: PCT/US02/36441

Related U.S. Application Data
(60) Provisional application No. 60/332,654, filed on Nov. 9, 2001.

(51) Int. Cl. A61B 1/06

(52) U.S. Cl. 600/105; 600/178

(57) ABSTRACT

Devices (1) and methods for accomplishing tasks within a body using infrared imaging are disclosed which, in connection with other known components, are useful in ablation, stitching and other operations, identification of sizes and composition of objects, and the creation of maps by taking multiple images at different positions or times.

This application claims the benefit of U.S. provisional patent application No. 60/332,654 filed on Nov. 9, 2002.

### BACKGROUND OF THE INVENTION

1. Field of the Invention

This invention relates to cardiac catheterization and real-time, forward imaging through blood.

2. Related Art

The following references provide useful information in the filed of the present invention, and are incorporated by reference herein:

| | | |
|---|---|---|
| Peronneau | November 1970 | 3,542,014 |
| Carpentier | April 1972 | 3,656,185 |
| Moulopoulos | June 1972 | 3,671,979 |
| Boretos | November 1977 | 4,056.854 |
| Cribier | October 1988 | 4,777,951 |
| Cragg | May 1990 | 5,085,635 |
| Fischell | June 1993 | 5,219,329 |
| Drasler | May 1995 | 5,370,609 |
| Laur | June 1995 | 5,399,158 |
| Avitall | January 1996 | 5,487,385 |
| Edwards | September 1996 | 5,546,662 |
| Lodder | September 1996 | 5,553,610 |
| Kotula | October 1996 | 5,569,275 |
| Swanson | December 1996 | 5,582,609 |
| Swartz | December 1998 | 5,846,223 |
| Shearon | July 1999 | 6,064,902 |
| Haissauguerre | May 2000 | 6,064,902 |
| Amundson | January 2001 | 6,178,346 |
| Suorsa | March 2001 | 6,206,831 |
| Yoshida | May 2001 | 6,226,076 |
| Tu | May 2001 | 6,238,390 |
| Tu | June 2001 | 6,241,727 |
| Gaiser | June 2001 | 6,241,728 |
| Tu | October 2001 | 6,303,133 |
| Stewart | December 2001 | 6,325,797 |
| Sutton | September 2002 | 6,443,950 |

Heart disease is the number one killer in the US and many other countries. In the United States, heart disease results in the death of almost one million people per year. The high mortality and morbidity rate has led to many drug and device therapies to intervene in the progression of heart disease. Aggressive therapy for many forms of heart disease involve interventions where a cardiologist inserts a catheter in the patients artery or vein and performs procedures such as angioplasty, pacemaker or implantable defibrillator lead insertion or electrical mapping. These procedures have grown dramatically on a cost-basis: 947 million dollars were spent in 1990, compared to 4.6 billion dollars spent in 1996.

Interventional procedures in cardiology are all the more remarkable since these procedures are performed only under fluorscopic guidance. Radiography presents the physician with a faint outline of the heart and its relation to the catheter. While fluoroscopy provides the cardiologist a crude guide, it does not allow examination of surfaces of the heart and vasculature or provide enough vision to guide procedures such as angioplasty or ablation.

In other body cavities, not filled with blood, such as the stomach or esophagus, fluid can be evacuated permitting visible wavelengths to be used in endoscope imaging. Visualizing the structure allows minimally invasive procedures such as ablating, stapling and suturing to be performed. These procedures, called laparoscopic procedures, are guided by the insertion of laparascope or an endoscope, permitting visual examination of the treatment. These procedures are done in either a clear fluid or in air and cannot be performed in the presence of blood. It is unfortunate cardiology has not had access to this technology since the common procedures would benefit from visualization.

The advantages to seeing structures in the cardiovascular system are numerous. Current methods of visualizing structures in the cardiovascular system are limited to fluoroscopy, ultrasound and angioscopy. Fluoroscopy, is the standard visual tool used to image interventional cardiology procedures. It is applied by a large X-ray apparatus on a C-arm that will rotate around the patient through 180 degrees. The heart appears as a faint outline; while the metallic catheters are brightest. This allows for gross estimation of the catheter end to faint landmarks of the heart. The C-arm is frequently repositioned to give better viewing perspectives. Once the catheter has been navigated to the heart, it can be placed in a coronary artery. In a self-contained entity such as an artery or vein, fluoroscopic sensitive dye can be injected out the distal end of the catheter and viewed on the fluoroscopy camera for a short distance before it diffuses with blood. This technique is used to spot constricted areas in the coronary arteries. It has been shown that radiography, however, usually underestimates the degree of stenosis and therefore is only useful in providing a gross measure of flow. Intraluminal and intracavitary ultrasound in which the ultrasound transducer is inserted into a cardiac chamber (intracavitary) or artery (intraluminal) provides a low-resolution, two-dimensional slice view of the cavity or artery interior. It is of little use in guiding procedures since it does not provide a direct forward-viewing image of the target.

In a recent patent (U.S. Pat. No. 6,178,346) by the same inventor and assigned to the same company, means of achieving direct vision through blood are disclosed using near-infrared light in wavelength regions where the absorption and scattering are at a minimum. The cardiovascular embodiments in U.S. Pat. No. 6,178,346 disclose a method and means of visualizing coronary artery plaque, viewing a catheter ablation procedure and viewing the placement of leads and catheters. This invention discloses the use of near-infrared vision in other applications and discloses more advanced techniques in the use of near-infrared endoscopy.

The purpose of the patent is twofold: disclose applications enabled by a near-infrared endoscope and disclose advanced techniques in near-infrared endoscopy Applications include catheter ablation, heart valve repair, and thrombus detection/removal. Advanced techniques include dynamic characterization of tissue elasticity, chemical sensing using near-infrared light, distance measurement with near-infrared endoscopy and arterial/vascular mapping.

Catheter Ablation

In the field of cardiology, arrhythmias (irregularities in heart rate) are increasingly being treated by a procedure called catheter ablation. In catheter ablation, a catheter is inserted, usually from the femoral veins, into the right heart of a patient, where it is critically positioned to ablate spots in the heart, thought to be propagating the arrhythmia. If successful, the arrhythmia is permanently disrupted and the patient no longer requires conventional therapy such as drugs, repeated cardioversion or the implantation of expensive defibrillators and pacemakers. For example, aberrant conduction pathways between atria and ventricles create some pathological high heart rates, called supraventricular tachyarrthymias. These pathways are detected by mapping electrical potentials with multi-electrode catheters in the atrium. Once located, a small radio-frequency burn of about 5-20 square millimeters is created in close proximity to the pathway.

More common arrhythmias such as atrial fibrillation, flutter and more lethal arrhythmias such as post-myocardial-infarct ventricular tachycardia require lines to be burned instead of “spots”. Atrial fibrillation is the most common arrhythmia, affecting over 3 million people in the United States. In this arrhythmia, the atria quiver, no longer pump blood, and there is an unstable heart rate as a side feature. Patients with AF are much more prone to stroke, congestive heart failure, myocardial infarctions and fatal ventricular arrhythmias. Patients can be in temporary (paroxysmal) atrial fibrillation or permanent atrial fibrillation (most dangerous). Atrial flutter often a precursor to atrial fibrillation, is a fluttering of the atria, also with loss of atrial mechanical function. It has a prevalence ranging from 1 in 81 to 1 in 238 hospitalized patients. This arrhythmia is usually disabling and resistant to antiarrhythmic drugs and it carries a potential risk of thromboembolism and chycardiomyopathy. Post-myocardial-infarct ventricular tachycardia (PMIT) occurs following a myocardial infarction. The infarct sometimes results in short-circuiting of the ventricular electrical activation pattern, resulting in tachycardia. It is a very lethal tachycardia and as a result is the principle indication for receiving an implantable defibrillator.

For these arrhythmias, ablation lines, rather than spots, need to be created to eradicate these arrhythmias, based on anatomical considerations rather than electrical potentials. For atrial fibrillation, circular lines around the pulmonary veins and sometimes-additional lines seem to be effective for the eradication of the arrhythmia. For atrial flutter, a linear ablation around the tricuspid annulus and Eustachian valve and ridge on the septum is effective in terminating the arrhythmia. For post-myocardial-infarct ventricular tachycardia, a circular ablation around the infarct is sometimes successful in eradicating the arrhythmia.

Since these procedures are performed without local visualization, the location of the burns cannot be seen, making connection of the spots very difficult. As stated in Lardo et al. in Visualization and Temporal/Spatial Characterization of Cardiac Radio frequency Ablation Lesions using Magnetic Resonance Imaging Circ 2000: 102:698-705, “Since its initial description in 1982, radio frequency ablation (RFA) has evolved from a highly experimental technique to its present role as first-line therapy for most supraventicular arrhythmias. More recently, the clinical indications for RFA have expanded to include more complex arrhythmias that require accurate placement of multiple linearly arranged lesions rather than ablation of a single focus. In contrast to catheter ablation of accessory pathways and atrio-ventricular nodal reentrant tachycardia, for which detailed mapping is necessary to identify appropriate sites for energy delivery, sites for catheter ablation of atrial flutter and atrial fibrillation, for example, are identified almost entirely on an anatomic basis. Although the feasibility of anatomy-based catheter ablations been demonstrated with standard catheter ablation techniques, these procedures are extremely time-consuming, require prolonged fluoroscopy exposure and have been associated with a high incidence of complications. For these reasons, there is general agreement that new approaches to facilitate anatomy-based catheter ablation are needed”

Catheter ablation of atrial fibrillation is currently accomplished by accessing the left atrium through a needle puncture from the right atrium, and placing circular lesions around the pulmonary veins. Various circular burn configurations have been evaluated, ranging from encircling all of the pulmonary veins to encircling each one individually. Some protocols also advocate the placing of additional linear lesions between the pulmonary vein and the cardiac valve. A dangerous complication of this procedure is stenosis of the pulmonary veins from ablations too far inside the pulmonary veins. Pulmonary vein stenosis can be disastrous and can lead to heart-lung transplantation.

Atrial flutter is the latest arrhythmia now being principally treated with catheter ablation due to recent identification of the “short-circuit” location. As stated in Nakagawa et. al. “Use of a three-dimensional, non-fluoroscopic mapping system for catheter ablation of typical atrial flutter”. PACE 21: 1279-1286 (1999) “Recent studies have shown that typical atrial flutter results from right atrial reentry around the tricuspid annulas and Eustachian valve and ridge on the septum. Creation of a complete line of conduction block across the subeustachian and the eustacean valve, eliminates typical and reverse-typical atrial flutter.” As in atrial fibrillation, it is very difficult to blindly make a continuous lesion. As stated in Jais, P et al in “Prospective randomized comparison of irrigated-tip catheters for ablation of atrial flutter”.Circ 101; 772 (2000),“Common atrial flutter designates a reentrant atrial arrhythmia with a stereotypical surface ECG showing continuous undulation with a saw tooth morphology in the inferior leads. The reentrant circuit has been shown to be critically dependent on conduction through the isthmus of the atrial myocardium limited by the tricsuspid annulus and the inferior vena cava. RF ablation of this isthmus, the only curative treatment for common flutter, is now widely performed and is the most common indication for ablation in some centers. Complete and bi-directional conduction block in the isthmus is the best end point for long term success. However, the creation of a continuous and transmural lesion along the 1-6 cm if the isthmus is sometimes difficult to achieve with current RF technology designed to punctate lesions.” Oftentimes, gaps in the ablation line can produce atrial fibrillation, a more dangerous arrhythmia.

Currently, ablation of PMIT is still an experimental procedure due to an inability to visualize the infarct location. There is surgical correlate to eradication of PMIT: ventricular aneurysmectomy. In these procedures the infarct is either removed or ablations are placed around the infarct, using cryosurgical tools or lasers. There have been experimental attempts to accomplish disruption of the short circuit using focal burns; however, this has been restricted to a minority type of PMIT (monomorphic PMIT accounting for <10% of PMIT patients). The more common polymorphic tachycardias are much more difficult to eradicate with small burns since eradication of one form of the tachycardia can lead to another different form with a different short-circuit pathway. Current procedures attempt to make small focal burns in or around the infarct, guided only by electric potentials. The basic strategy is to locate and ablate a small isthmus within the infarct which is critical to maintaining the short-circuit. A more ideal approach would be to recognize the infarct boundaries and ablate around them mimicking the surgical procedure of ventricular aneurysmectomy.

Various ablation catheters have been developed which produce continuous lesions.

Avitall (U.S. Pat. No. 5,487,385), Kroll (U.S. Pat. No. 6,287,306), Tu (U.S. Pat. No. 6,238,390) and Shearon (U.S. Pat. No. 6,064,902) disclose catheters capable of producing linear lesions. Sutton (U.S. Pat. No. 6,443,950) and Swartz (U.S. Pat. No. 5,846,223) disclose catheters, which make continuous lesions for atrial flutter eradication. Catheters capable of forming linear circular lesions, needed for pulmonary vein isolation, are disclosed by Haissauguerre (U.S. Pat. No. 6,064,902), Tu (U.S. Pat. No. 6,241,727), Stewart (U.S. Pat. No. 6,325,797) and Gaiser (U.S. Pat. No. 6,241,728). All of these catheters rely on spatial configurations to orient the catheter in close proximity to the targeted the tissue and electrode separations small enough so that the individual lesions form one continuous lesion. For example, Stewart (U.S. Pat. No. 6,325,797) teaches a catheter of closely spaced electrodes where the distal end assumes a circular configuration for placement around a pulmonary vein.

In general, these linear-lesion producing catheters have two problems: variations in cardiac anatomy and inability to assess lesion production. If the cardiac area to be ablated conforms to the shape of the lead and all of the ablation electrodes are in intimal contact with the tissue, a linear lesion at the proper location should be formed. However, there is great variation in cardiac anatomy among patients. For example, most patients have four pulmonary veins, however some patient's have more veins. Some patients have pulmonary veins in close proximity to each other rather than being spatially separate. If a circular configured catheter, such as Stewart (U.S. Pat. No. 6,325,797), were used in pulmonary veins which are contiguous to each other, some of the electrodes might actually reside in the neighboring pulmonary vein, possibly causing pulmonary vein stenosis.

Producing a continuous lesion by connecting individual spot lesions is also somewhat speculative, since the contact pressure against tissue determines the size of the lesion. Catheter configurations such as Swanson (U.S. Pat. No. 5,582,609), which form a linear lesion from the connection of small circular lesions, use electrode separations, that produce a linear lesion if the electrodes are lying against tissue. If an electrode is not lying against tissue, a much smaller lesion or no lesion will be formed, leaving a corresponding gap in the linear lesion. Gaps in linear lesions may actually worsen the arrhythmogenic condition, such as in atrial flutter ablation, where gaps in the lesion can lead to atrial fibrillation. In an effort to verify tissue contact, Suorsa (U.S. Pat. No. 6,206,831) discloses a sensitive ultrasound means of evaluating tissue contact by having the ultrasound transducers adjacent to each of the electrodes. The patent assumes that if the electrodes have a certain separation and the ultrasonic transducers verify tissue contact, then a continuous lesion will result.

The difficulty of making continuous lesions with radio-frequency energy has led to the exploration of other ablation sources. Sources such as lasers, microwaves, ultrasonic energy and freezing have all been proposed by investigators as a means of making linear lesions. The safety and efficacy of these approaches is still unclear. For example, laser ablation is a common technique in other areas of medicine, where it is possible to image the effects of the ablation. When it is performed blindly, however, laser ablation can lead to perforation of a cardiac chamber. In fact, one of the many positive attributes to radio-frequency energy is that it can be applied safely since ablation is limited to about one millimeter from the surface of the electrode.

Heart Valve Repair

The circulatory system consists of a heart, blood vessels and four valves, which regulate the pumping cycles of the heart. These four valves include on the right side of the heart, the tricuspid valve separating the right atrium from the right ventricle and the pulmonary valve separates the right ventricle from the pulmonary artery. On the left side of the heart, the mitral valve separates the left ventricle form the left atrium while the aortic valve separates the left atrium from the aorta. Cardiovascular function is reduced if any of these four valves do not open or close properly. With aging, valves can change configuration to states where the leaflets no longer fully close due to changes in the shape of the valve annulus or the valve becomes stenosed from calcification. When this occurs, the valve often needs to be replaced with an artificial heart valve or the existing valve is repaired. Most heart valve repair requires chest surgery, either open-heart in which the patient is placed on cardiopulmonary bypass or a minimally invasive technique where small incisions are made for the passage of tools in the chest to perform the procedure.

It has been a long-term goal to be able to do valve repair and introduce artificial valves percutanoeusly using a catheter introduced into a vein or artery. This goal has been difficult to attain, since there is no real-time imaging modality currently available which provides a view of the valve leaflets. The imaging modality available to view valve function is echocardiography, where ultrasound transducers placed on the chest create a slice image. Although it does provide information about valve function, it does not provide a view of the valve leaflets needed to repair or replace a valve. Currently, a valve procedure, not requiring a chest operation, is a procedure called valvuloplasty (U.S. Pat. No. 4,777,951), where a balloon is inserted in the valve and expanded with saline to create a larger valvular opening, alleviating valvular stenosis. As can be appreciated, this procedure requires no imaging since it uniformly expands the valve.

There are other catheter procedures disclosed in the patent literature, which attempt to introduce an artificial valve, using a catheter. Moulopoulos (U.S. Pat. No. 3,671,979) describes an umbrella-type valve, which is inserted through a catheter placed in the cardiovascular system. Boretos (U.S. Pat. No. 4,056,854) describes an artificial aortic valve catheter, which can be used to insert a valve through a catheter procedure. These and other approaches to introduce an artificial valve using a catheter have not gained acceptance since the valve introduction process cannot be imaged. Concerns include insufficient anchoring since the valves are not sutured in place, interference with the existing valve and leaks around the valve periphery, which can lead to thrombus formation and improper valve placement.

There are a variety of valve repair techniques performed by cardiac surgeons in open-chest procedures, which improve valve function. There is a procedure called the “butterfly” procedure, in which the mitral valve has the leaflets stitched in the center of the valve so that the valve opening and closing resembles the flapping of butterfly wings. This procedure reduces regurgitation, and the patient has improved valvular function, without requiring an artificial valve. It would be highly desirable to perform this procedure through a catheter—if the mitral valve could be visualized real-time during the procedure.

Many valvular defects are associated with dilatation of the valve annulus preventing complete closure by the valve leaflets. Oftentimes a ring is placed around the heart valve to improve its function in chest surgery, a device called a valvuloplsty ring. This ring provides annular support for the heart valve, thereby improving its function. Carpentier (U.S. Pat. No. 3,656,185) provides disclosures of this technique. A foldable version of a valvuloplsty ring has been proposed, whereby the ring is inserted into a catheter, deployed out of the catheter and oriented to proper position and attached to the valvular orifice, thereby eliminating chest surgery. Direct real-time imaging would be useful in the orientation and attachment aspects of the procedure.

Another means of addressing valve dilation, is to heat the valve annulus, thereby causing shrinkage and improved apposition by the valve leaflets. Heat application has been described by Edwards (U.S. Pat. No. 5,546,662) and Tu (U.S. Pat. No. 6,303,133). The device by Tu involves the introduction of a catheter-based circular heating element, designed to fit on the valve annulus and heat the entire annulus. Here to, direct real-time imaging would be useful in directing heating elements to the valve annulus rather than relying on the heating element geometry to gain apposition to the valve annulus. In addition, viewing the valve annulus would permit applying the heating to selected portions of the valve annulus, which would most benefit leaflet closure.

Thrombus Detection and Removal

A thrombus is a mass of fibrin and red blood cells, which can block the flow of blood if it becomes lodged in an artery or vein. The most common condition involving thrombi is deep vein thrombosis, which can lead to pulmonary embolism and possibly death. Deep-vein thrombosis is a common illness resulting in suffering and death if it is not treated properly. It tends to occur most often in patients who are not ambulatory such as bed-ridden or wheelchair bound patients since the lack of leg exercise or movement greatly exacerbates the formation of a thrombus. It affects ambulatory patients as well, particularly pregnant women, where it is the greatest cause of death during childbirth. Deep-vein thrombosis occurs in about 2 million Americans each year. Death can occur quickly if a venous thrombi breaks of to form a pulmonary embolism. The thrombus blocks the passage of blood to the lungs. If it substantially blocks blood flow, immediate death will frequently occur. About 600,000 Americans develop pulmonary embolism with 60,000 dying from the complication.

There are three methods used to diagnose deep-vein thrombosis. Venography is a technique whereby a radio-opaque dye is injected into the foot where it flows towards the heart. Viewing a fluoroscopic image will reveal a deep-vein thrombosis. Impedance plethysmography is performed by placing two sets of electrodes on the patient's leg to measure blood flow and placing the leg in oversized blood pressure cuff. The cuff is inflated to obstruct the return-blood flow. When deflated, the time is measured for the venous return back to the heart. If there are delays in venous return, the presence of a deep-vein thrombosis is revealed. Finally, ultrasound imaging is also employed. Here an ultrasound probe is place over the common femoral artery in the groin under gentle pressure and moved distally towards the foot. The criterion for deep-vein thrombosis is non-compressibility of the venous lumen under gentle probe pressure.

Pulmonary embolism is diagnosed using fluoroscopic techniques. Pulmonary angiography in which a radio-opaque dye is infused in the pulmonary vein and viewed fluoroscopically is the gold standard. However, this equipment is not readily available in hospitals, and so most hospitals take a lung X-ray to rule out the presence of a pulmonary embolism. This is rarely diagnostic. Sometimes a semicircular opacity can be found which is strongly suggestive of pulmonary embolism. Other radiographic features compatible with pulmonary embolism include pleural effusion, raised hemidiaphragm and various vascular shadows on the x-ray.

Since it is not possible to see the thrombus directly, systemic drugs are applied to reduce the thrombus size. Currently, treatment for deep-vein thrombosis and pulmonary embolism is high-dose heparin infusion. Heparin is an anticoagulant, which reduces thrombus formation. The principle complication of this therapy is internal bleeding.

There are various techniques for extracting or dissolving thrombi. Techniques vary from mechanically removing the clot to lysing it with chemicals or applying pressure-inducing means. Cragg (U.S. Pat. No. 5,085,635) describes an infusion catheter to infuse drugs such as urokinase into the thrombus. U.S. Pat. No. 5,370,609 discloses a technique to emulsify them with a high-pressure saline flush. U.S. Pat. No. 5,569,275 discloses a mechanical thrombus maceration catheter device. Laur (U.S. Pat. No. 5,399,158) describes an ultrasound-based technique of lysing thrombi. Fischell (U.S. Pat. No. 5,219,329) describes a two-piece sheath means of extracting thrombi. Ritchie in Circulation vol 73, 1006-12 describes a rotational auger device, which winds the thrombus into a central shaft.

The above techniques do not rely on imaging the thrombus for its removal. If direct, real-time imaging were available, a broader range of techniques would be possible.

Dynamic Characterization of Tissue

U.S. Pat. No. 6,178,346 discusses and makes claims for illuminating structures obscured by blood with infrared illumination and recording the reflected image in an infrared camera. Illumination wavelength candidates should be in a local absorption mimimum such as: 800-1350 nm, 1550 nm-1850 nm and 2100 nm-2300 nm. There is no discussion in the patent regarding using sequential images to determine the dynamic characteristics of the structure of interest. The two areas of greatest importance in cardiology where the dynamic characterization of tissues is most important is in ischemic plaque recognition and identifying myocardial infarct regions.

Coronary artery plaque varies form rigid calcified deposits to soft, fibrous tissue plaque consisting of a thin capsule covering a fluid-filled interior. It is now recognized in the cardiology community that most serious heart attacks and strokes are due to this type of plaque formation, which is called “vulnerable plaque”. Vulnerable plaque consists of a thin fibrous capsule containing a gelatinous fluid consisting of lipids and blood cells. When it ruptures (usually due to emotional or physical stresses), the released fluid can cause massive coagulation. If a vulnerable plaque ruptures in the coronary arteries, it can lead to a massive heart attack; in the carotids, a massive stroke. “The rupture of a plaque will be the cause of death of about half of all of us in the United States,” says Dr. Steven Nissen of the Cleveland Clinic in a 1999 Associated press article by Daniel Haney (Assoc Press Jan. 11, 1999). “Understanding why they rupture is probably the most important question today in cardiology and even the most important question in all the country.” As stated in Stroke (Hatsukami, T S, Ross, R, Nayak, P L, Yuan, C. Visualization of fibrous cap thickness and rupture in human atherosclerotic carotid plaque in vivo with high-resolution magnetic resonance imaging. Stroke (2000) 112: 959-964) “Cardiovascular disease is the leading cause of death in the United States and greater than 70% of these deaths are related to atherosclerosis. Greater than 75% of the major coronary events were precipitated by atherosclerotic plaque rupture.”

One of the current interests in cardiology is finding regions of vulnerable plaque. Since vulnerable plaque of a thin capsule containing a lipid fluid, it has different dynamic movement then either calcified or fibrous plaque. As the pressure builds, threatening plaque rupture, the dynamic conditions continue to change as the capsule becomes more rigid. In intraluminal ultrasound (IVUS) there is a technique called elastography where a pressure pulse is applied down the IVUS catheter, while it is collecting sequential images. By comparing sequential images before and during the pressure pulse, an estimate of the strain on the tissue of interest can be made. The inherent low-resolution (100 microns) and the difficulty of making rapid sequential images make this technique inaccurate.

The other area where dynamic characterization of tissue would be of great interest would be in identifying infarct regions in the heart. When a heart attack occurs, a coronary artery is blocked and insufficient blood perfuse the region of the ventricles fed by that coronary artery. As a result, a portion of the ventricle undergoes cell death and no longer contracts like the uninjured muscle fibers. Currently, the presence of a myocardial infarct is determined chemically and sometimes by ultrasound if the infarct is large enough for detection. However, infarct boundaries cannot be determined with these techniques.

Chemical Sensing Using Near-Infrared Light

It is well known that certain biological materials and chemicals have identifiable absorption variations in the near-infrared spectrum. It is an established forensic technique for the detection of trace organic compounds. In the wavelength region between 1300-3000 nm, many organic compounds exhibit characteristic signatures (variation in absorption levels). In this invention, only compounds, which have signatures in the low-absorption water windows, as identified by the Amundson patent (U.S. Pat. No. 6,178,346), are of interest. Lipids are of special interest since they constitute the pool inside the vulnerable plaque and are within the water window of 1500-1850 nm. The have two signature wavelengths which occur at 1700 nm and 1760 nm. In addition, other chemicals, such as cholesterol, have signatures in this water window as well.

Lodder (U.S. Pat. No. 5,553,610) describes an acoustic resonance, near-infrared spectroscopy means of identifying certain biological material such as cholesterol and lipoproteins. As with other spectroscopy systems, wavelengths spanning the near-infrared spectrum are used. Such a technique would not be possible making spectrophotometric measurements through blood since many wavelength regions are too absorptive. In addition, these are very sensitive measurements involving an interferometer where any scattering, such as would be caused by intervening blood, would also be prohibitive of spectrophotometric measurements.

Distance Measurement with Near-Infrared Endosocpe

The Amundson patent (U.S. Pat. No. 6,178,346) demonstrates the usefulness of the technology in imaging plaque in the coronary artery. There is no discussion in the patent regarding making measurements of the size of objects in the field of view. Knowing the distance of objects in the field of view is of interest particularly in angioplasty procedures, where the physician is trying to determine the proper sized stent for placement in the artery. In intravascular ultrasound, these measurements can be made from determining the transit time for the ultrasound echo. Knowing the speed of sound, this can be translated into distance measurements of the object of interest. Peronneau (U.S. Pat. No. 3,542,014) discusses these techniques as applied to determining the diameter of coronary arteries.

Similar techniques are available for light transmission, such as Yoshida (U.S. Pat. No. 6,226,076). But due to the high velocity of light and the short distances traveled in the coronary artery (3-5 mm), it is impractical to measure such short transit times with conventional equipment.

Arterio/Vascular Mapping

Today, the arteial-venous tree is viewed on fluoroscopy with or without dye infusion and used for guidance during catheter introduction. The image is very feint (really a shadow) and provides no information of the catheter interior. Trans-Blood-Visualization (U.S. Pat. No. 6,178,346) provides local images of the coronary arteries. While local images are useful, it would be desirable to have a macroscopic view of the entire arterial-venous tree, as is currently available with fluoroscopy. It is an object of this disclosure to develop such a macroscopic image of the arterial-venous tree based on local images and measurement of catheter position in the vascular tree.

### SUMMARY OF THE INVENTION

This invention provides methods and means to apply near-infrared endoscopy to the following catheter-based procedures: linear ablations for the elimination of arrhythmias, heart valve repair or replacement and the detection and removal of thrombi. In addition, the invention discloses the following advanced techniques in near-infrared endoscopy: characterization of tissue elasticity, chemical sensing using near-infrared light, distance measurements with near-infrared endoscopy and arterial/venous mapping.

In catheter ablation, the present invention provides a method and means for near-infrared-guided catheter ablation of linear lesions. The near-infrared imager consists of a fiber-optic bundle about one millimeter in diameter, which can transmit near-infrared light. This bundle is connected on the distal end to a lens assembly, which spreads the light over a 30-90 degree cone. The proximal end is inserted into an interface cable, which contains and routes the near-infrared light source and the near-infrared camera. This viewing system provides direct real-time images of an area about 1-2 centimeters in diameter—wide enough to see multiple-lesion formation and to assess the continuity of linear lesions. The viewing system needs to be around a centimeter from the ablation point to record images of the ablation lesions. As the catheter assembly is implanted near the structure to be ablated with a linear lesion, the near-infrared imaging produces images of the surrounding tissue, permitting the physician to guide the catheter assembly to the precise anatomical location.

For example, if it is desired to place lesions around a pulmonary vein, the catheter's position relative to the pulmonary vein can be assessed. If the catheter is imaged to be in a position outside the vein, ablation can commence on the pulmonary vein. This avoids the complication of possibly producing pulmonary vein stenosis, by ablating inside the vein. If conventional radio-frequency energy and ablation electrode is used for ablation, the near-infrared image is used to assess whether the tissue is in contact with the ablation electrode and ablation proceeds. After burning the first spot, it is visualized on the near-infrared image and the catheter is moved to a position immediately adjacent the burn, permitting the second burn ot be contiguous with the first ablation. In this manner, contiguous linear ablations can be made in any shape or pattern at the proper anatomical landmarks.

Linear radio-frequency lesions can be generated easily either by “connecting the dots” with conventional ablation electrodes or by orienting a modified ablation electrode so that the electrical surface is only substantially touching tissue. Since the ablation electrode can now be seen on the near-infrared image, the electrode can be oriented so that only the active electrical surface is touching tissue. Normally, the hemispherical ablation electrode burns tissue and blood as well, which creates coagulum from the burning of blood by the electrode. For example, an “L-shaped” electrode could be constructed with all but one surface electrically insulated. The “L-shape” would be visible in the near-infrared image, and the uninsulated portion of the electrode could be oriented against tissue. If the electrode is only heating tissue, a longer and deeper lesion can be produced. without producing coagulum formation.

Besides orienting radio-frequency ablation electrodes against tissue, near-infrared visualization can also be used to orient other ablative energy sources. For example, laser ablation is out of favor since there is no visual feedback of where the laser is pointed.

Misdirecting the laser at structures like the free wall of the atrium can produce perforation with deleterious side-effects. If the orientation of the laser tip to the tissue is imaged, confirmation of appropriate positioning can be determined, prior to laser firing. Moreover, the lesion production can be viewed in real-time. Similarly, catheters using other ablative sources such as microwave energy, ultrasound and freezing and others can also be directed to an appropriate position relative to the structure, which needs to be ablated.

This invention can be embodied in several forms. The near-infrared viewing system can be integrated in a separate catheter in close proximity to the ablation catheter, a guiding catheter for passage of the ablation catheter or an integrated catheter where the ablation electrodes and the near-infrared imaging system are together in a composite catheter.

The invention also discloses method and means of guiding catheter-based heart valve repair and replacement using near-infrared imaging to guide the procedure. One of the embodiments is a procedure where the butterfly operation is accomplished using a catheter containing the near-infrared imaging and a working channel for the passage of a suturing mechanism. The catheter is inserted in the venous system, where it is routed to the right atrium and pushed through the left atrium using a needle puncture technique and oriented in a position is opposition to the mitral valve. The suturing mechanism is advanced until it is viewed to be touching the valve when closed. The valve leaflets can be held together by another tool or the suturing can occur during natural valve motion, always guided by the near-infrared imager. In the embodiment, one leaflet is first punctured by the needle followed by puncture of the other leaflet. As the suture tie is pulled together from the proximal end of the catheter, the valve leaflets will be joined and the valve will assume a butterfly configuration with the two leaflets sutured together at the center of the valve.

For patients with a dilated valve annulus, near-infrared viewing can be used to guide the insertion of an annuloplasty ring through a catheter. Imaging is needed in this procedure, since the ring must be first seated in proper position and securely attached to the valve annulus. The ring must be checked after the procedure with the near-infrared imager to insure that there is no leakage around the ring. The ring is folded inside the working channel where it is deployable by advancing it on the proximal end of the catheter. Once deployed and positioned over the annulus it is sutured in place. The suitability of the suturing is assessed by the near-infrared imager insuring there is no leakage around the ring.

Valve dilatation can also be accomplished by heating the valve annulus. Viewing the valve annulus with the near-infrared imager permits the heating element to be laid directly against the valve annulus. As heat is applied to a section of the annulus, leaflet closing is assessed to see if the leaflets are now touching during valve closure. If not, another section of the annulus is identified and heat is again applied until it is observed that the leaflets seal properly during valve closure. This iterative process of heating and evaluating leaflet closure can be performed iteratively until optimal valve closing is achieved.

The near-infrared imaging catheter also enables the removal of thrombi from veins or arteries. A two-lumen catheter contains the near-infrared imaging system and a working channel for the passage of tools. The thrombus has the appearance of a large spherical object. Once the thrombus is visualized, a tool is extended out of the distal end of the catheter. There are a variety of tools, which can remove a thrombus, if it can be visualized during the extraction procedure. They include suction, lysing, high-pressure saline flush and mechanical means. The embodiment presented uses an auger device for rotating the fibrin strands in the thrombus proximally through the catheter. This procedure is enhanced by near-infrared vision, since the thrombus moves while being augered, much like a ball of yarn unraveling. Moreover, the fibrin strands are fragile and break as they enter the catheter. Repeated application is often required. When the thrombus is visualized, re-applying the auger device is a straight-forward procedure since the auger can be oriented against the center of thrombus on each re-application.

Besides the applications, several advanced techniques in near-infrared endoscopy are disclosed. Advanced techniques include dynamic characterization of tissue elasticity, chemical sensing using near-infrared light, distance measurement with near-infrared endoscopy and arterial/vascular mapping.

With infrared illumination, comparing sequential images and highlighting regions, which change shape during pressure application, can derive a measure of strain. Because of the inherent resolution (10 microns) of light and rapid image acquisition times (up to 100 frames/sec), detailed pictures can be taken every 10 milliseconds. As the heart beats, a pressure pulse is experienced by the artery/vein/chamber. If a series a pictures are recorded and analyzed for moving sections near the end of the pressure pulse and immediately after, structures which are quivering or shaking can be identified by comparing images Fatty plaque will generate greater elasticity then calcified plaque during positive pressure changes. Following the pressure pulse, the fatty plaque images will show variation due to movement, while the calcified plaque will remain unchanged. The mechanical pressure from the beating heart is used as the pressure change in the embodiment for an estimate of strain. The pressure pulse can also be generated artificially using a transducer on the distal end of the catheter. The strain of a structure can then be displayed as a color or highlighted image overlaid over the real time image of the structure in question.

Using the same technique of comparing sequential images and finding moving areas, regions of infarct in the ventricles can also be determined. As the heart contracts, each individual heart muscle undergoes contraction, except those cells which no longer function due to a heart attack. These infracted cells do not contract. By comparing images during contraction, regions, which move the least are possible infarct areas. Those areas, not moving to the same degree as the rest of the heart chamber, could be highlighted or false colored for identification.

This invention also discloses means of determining the size and distance from the endoscope for structures of interest in the field of view. Sizes and distances are determined from using a triangulation method. The catheter translational movement is determined from a device affixed to the on the proximal end of the lead introducer. The device is an optical reader, which can detect marks on the catheter. As the catheter is advanced the reader determines the position of the catheter on the proximal end. If a distance or size needs to be determined, the physician marks the structure on the video monitor. The near-infrared system computer goes back in memory and measures the dimensions of the same structure. Knowing the distance traversed and the change of object size, a distance or structure size is determined using triangulation techniques.

Lastly, the invention discloses a means of creating a computer generated 2 or 3D map of the vascular tree. Since the catheter position in the vascular tree is known from the reader at the proximal end, and images are taken every 10-40 milliseconds, a series of internal images of the vein or artery and the corresponding catheter position is available for the computer to create a 3D map of the vasculature traversed by the catheter. Starting with a typical vascular tree stored in memory, the computer adjusts the parameters based on the individual pictures. The output is the vascular tree of the patient with proper adjustments for bifurcations, diameter and size. For example, if a physician is interested in the plaque formation in coronary arteries, he can view the interior of a selected artery and could view the internal endoscopic images made at that point.

In this manner, a physician could evaluate the progression of plaque formation in between visits. A near-infrared catheter is routed through the coronary arteries of interest and compared with previous visits. Since the computer knows the position of the catheter in the vasculature and the corresponding internal images, plaque regions, which have changed over time, could be presented to the physician.

### BRIEF DIESCRIPTION OF THE DRAWINGS

FIG. 1 is a schematic of the near-infrared endoscope system.

FIG. 2A is a view of the distal end of an embodiment where the imaging catheter and the ablation catheter are separate.

FIG. 2B is the view of FIG. 2A as seen by the near-infrared endoscope.

FIG. 3 is a view of the distal and proximal ends of an imaging catheter configured in a two-lumen catheter with a working channel for the introduction of the ablation catheter.

FIG. 4A is a view of the distal end of an imaging catheter configured in a two-lumen catheter with a working channel for the introduction of a directable L-shaped ablation catheter.

FIG. 4B is the view of FIG. 4A as seen by the near-infrared endoscope.

FIG. 5 is a view of the distal end of a catheter for heart valve repair using a stapling technique to join the valve leaflets at the center.

FIG. 6 is a view of the distal end of a catheter used for identification and removal of a thrombus.

FIG. 7 is a graph of the picture acquisition times with respect to the pressure curve of the chamber.

FIG. 8 is a graph of the absorbance spectrum of blood and lipids

FIG. 9 is a schematic of a two-wavelength near-infrared system.

FIG. 10 is a view of the proximal end of a catheter capable of measuring distances and diameters of objects seen on the distal end.

FIG. 11 is a schematic of a vascular map measured by a near-infrared endoscope.

### DETAILED DESCRIPTION OF THE EMBODIMENTS

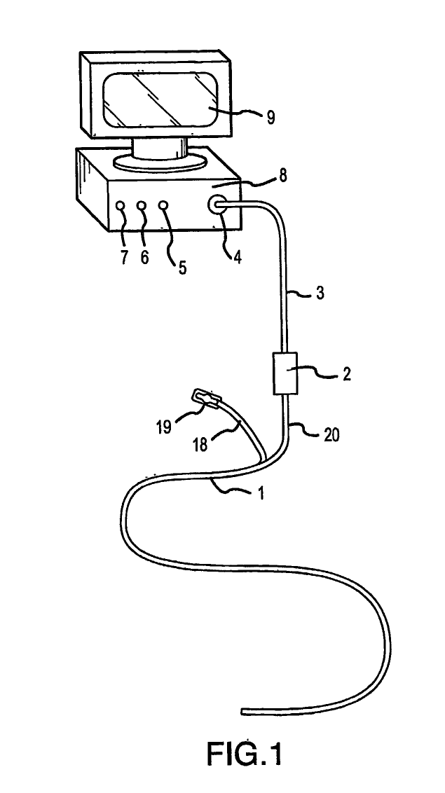

FIG. 1 shows the near-infrared imaging endoscope system. The system consists of a near-infrared endoscope (1). The endoscope (1) bifurcates into two segments, one branch (18) containing the wires for the articulation mechanism goes to a handle (19) with a control to articulate the catheter distal end. The bifurcation (20) contains the optical fibers, which are connected to an interface box (2) containing the light source and imaging sensor. The fiber assembly consists of illumination and imaging fibers with lenses placed on both ends of the catheter. A cable (3) to the near-infrared imaging acquisition unit (8) [we don't use that term in old patent] as described in U.S. Pat. No. 6,178,346, connects to the interface box (2). The acquisition unit (8) contains the system controller and image processing software and imaging controls (5, 6, 7). The acquisition unit (8). The details of the infrared-imaging are described in U.S. Pat. No. 6,178,346 and thus need not be repeated in detail herein in connection with any of the embodiments. Briefly summarizing that patent, the catheter 1 houses an optical head assembly which, in connection with a light source, imaging sensor, and associated components enable infrared catheter imaging.

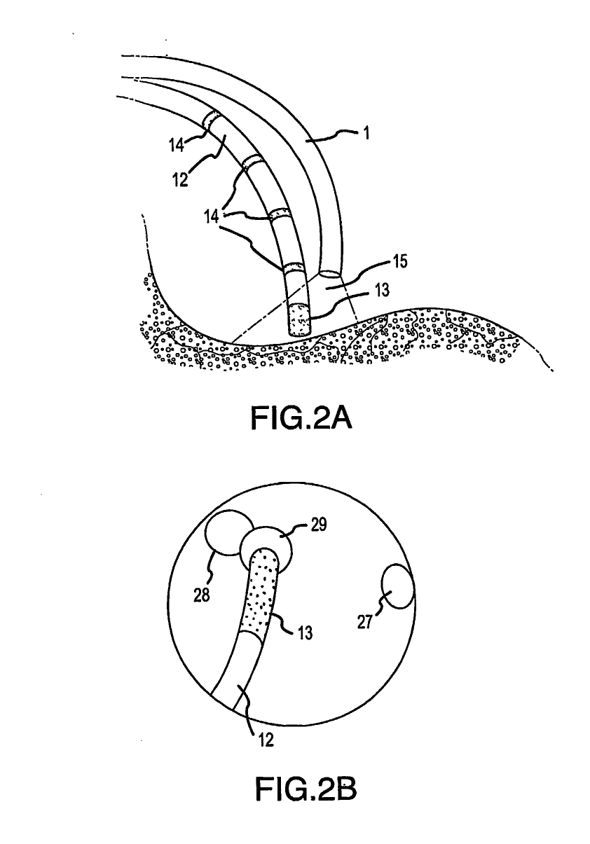

The first embodiment is a configuration where the near-infrared imaging catheter is separate from the ablation catheter. FIG. 2A shows an ablation catheter (1) placed on the surface of the right atrium (11) for the purpose of ablating the isthmus for eradication of atrial flutter. The ablation catheter (12) on the distal end has a series of four ring electrodes (14) and terminates in a hemispherical ablation electrode (13). The near-infrared endoscope (1) is within one centimeter of the ablation electrode (13) and has a field of view (15) of 90 degrees. The ablation catheter (12) is maneuvered until the ablation catheter is in position over the target tissue. Prior to the ablation, the imaging catheter captures images of the ablation catheter (12) and the tissue surface. If the ablation electrode (13) is touching the surface of the endocardium (11), as seen on the near-infrared monitor, ablation can proceed. After verification of tissue contact, a radio-frequency burn (i.e, RF energy) is applied to the distal ablation electrode. FIG. 2B is the near-infrared image as seen by the near-infrared endoscope after the creation of the radio-frequency lesion. The ablation catheter (12) has just finished creating a lesion (29) after making two other lesions (27, 28). The burn is imaged, and if adequate, the ablation catheter is moved to a position adjacent to the burn using feedback from the near-infrared imaging system. In FIG. 2B, the lesion (29) is made so that it is connected to an earlier-made lesion (28). If more connecting lesions are made, right of lesion 16 on FIG. 2B to lesion 27, a line extending from lesions 27-28 will be formed. In such a manner, a linear lesion of any configuration can be made on tissue anywhere in the heart.

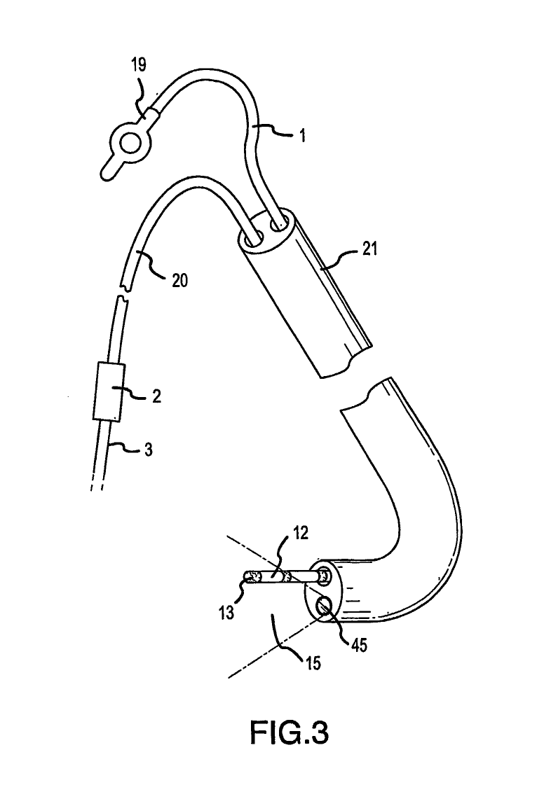

FIG. 3 shows the distal and proximal ends of a two-lumen catheter (21) where one lumen contains the illumination and imaging fibers and the other lumen is a working channel where a radio-frequency ablation catheter (12) is inserted. Placing the near-infrared imaging assembly in an introducer with a working channel keeps the ablation electrode in close proximity to the field of view (15), which is typically between 30-90 degrees. Smaller field of views are possible with this approach since the imaging assembly can be mechanically constrained to view the expected position of the ablation catheter. The working channel permits insertion of any type of ablation catheter, including those using other energy sources. Energy sources reported in the literature which ablate tissue or produce cell death include the following listed according to type of injury produced and usefulness of near-infrared imaging:

| | Ablative/Causes Cell | Lesion |
|---|---|---|
| Energy Source | Death | Characteristics |
| Lasers-light energy | Ablative | Crater formation, |
| | | sometimes burn appearance |
| Cryoablation- | Cell Death | Crystalline |
| Freezing | | formation, tissue does not |
| | | contract |
| Microwaves | Ablative | Crater formation |
| Ultrasound | Cell Death | Tissue does not |
| | | contract |
| Chemical injection | Cell Death | Tissue does not |
| | | contract |
| Heat energy | Ablative | Crater formation, |
| | | sometimes burn appearance |

Any catheter employing these and other electrical conduction-disrupting energy sources can be inserted in the working channel and viewed in the near-infrared imager. The ablation procedure would be similar to the radio-frequency energy ablation catheter used in this embodiment.

In FIG. 3, the proximal end of the two-lumen catheter has a radio-frequency ablation catheter (12) inserted into the lumen. The ablation catheter terminates in a handle (19), which has a control for deflecting the catheter. On the distal end of the catheter (21) the ablation catheter (12) is seen emerging. The ablation electrode (13) is extended between 0.5-2.0 cm from the catheter (21), in the field of view (15) of the near-infrared imager. The proximal end of the near-infrared imaging fiber bundle (20) is inserted into an interface box (2), which transmits optical and electrical signals to the near-infrared imager system.

The two-lumen catheter (21) is inserted in the vicinity of the site to be ablated. The ablation catheter (12) is pushed out to a position between about 0.5-2.0 cm, depending on the field of view (15) of the near-infrared lens on the distal end of the catheter. The ablation electrode (13) is directed by deflecting it with the controller on the handle (19) until it is seen to be touching the target tissue. Radio-frequency energy is applied, leaving behind a small crater, which is visible on the near-infrared imager. The ablation catheter (12) is then further deflected so that the catheter is adjacent to the crater produced from the first radio-frequency application. A second burn is applied and the second produced crater is viewed to see if it is contiguous with the first crater. By repeating this procedure, a linear lesion in any orientation can be created anywhere on an anatomical structure viewable by the near-infrared monitor.

Since the positioning of the ablation electrode, with respect to the tissue, can be viewed on the near-infrared imager, the electrode can be oriented in various orientations. This permits the development of a radio-frequency electrode, which is mostly electrically insulated so that the active electrical surface can positioned against tissue. This would produce deeper lesions and coagulum and would require less energy since much less blood is being heated then with hemispherical electrodes. A hemispherical electrode ablates mostly blood as well as tissue since a minority of the surface is touching tissue. For example, if a hemispherical electrode is touching tissue with 20% of its surface area, 80% is touching blood. Blood is about ⅓ less resistive then tissue. This means that only ⅓×20%=6.6% of the energy is directed towards tissue ablation. The remaining 93% of the radio-frequency energy heats the blood, causing coagulum formation and the possibility of embolic injury in the patient. If a mostly insulated electrode were used, with one electrically active face, the face could be oriented so that it is in direct contact with the tissue. If the electrode was in minimal contact with blood, a 15 fold improvement in efficiency would be realized. Less radio-frequency energy (1/15) would need to be applied to this electrode for comparable lesion formation as with the hemispherical electrode. With this improved geometry, either longer or deeper lesions could be formed with the same energy used for hemispherical electrodes. Lesions around one centimeter in length could be formed from the same energy required to make 1-2 mm lesions with hemispherical electrodes.

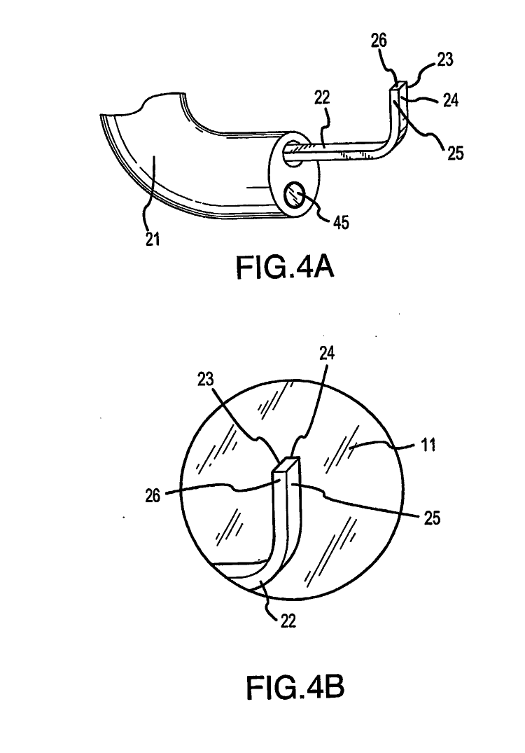

FIG. 4A shows an L-shaped, rectangular ablation electrode (22), which is inserted into the two-lumen catheter (21). The L-shaped electrode has the face (24) opposite the two-lumen catheter electrically active. The other three faces (22, 23, 25) insulated with an electrically insulative material such as parylene or silicone rubber. FIG. 4B is the image as seen on the near-infrared monitor. The distal portion of the ablation electrode (25) can be visualized as well as which surface is in contact with the tissue (11). The electrode is pushed out of the catheter about 0.5-2.0 cm so that the electrically active surface (24) is contacting the tissue to be ablated. Radio-frequency energy is applied and a long linear lesion is created, giving the appearance of a cratered line. The electrode could then be manipulated or rotated to make a second lesion connecting with the linear lesion made in the first application of radio-frequency energy. In this manner, a long ablation line could be formed with just a few burns. Also, the chance of lesion gaps would be greatly reduced since each ablation is a line rather than a spot lesion.

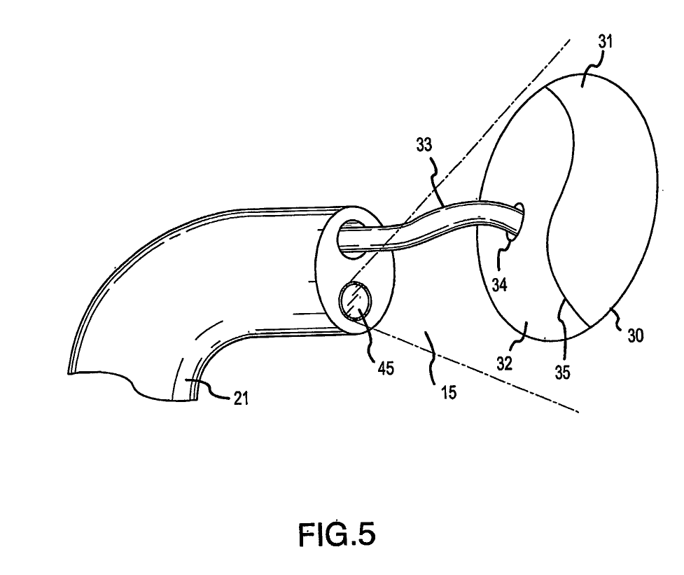

FIG. 5 is an embodiment of a catheter (21), which applies a butterfly stitch for repair of a mitral valve (35). The mitral valve is located in the left heart, so access to the left heart is achieved by transeptal puncture from the right atrium into the left atrium. A sheath is placed over the transeptal puncture needle providing a conduit from the left atrium to the entry vein. A two-lumen catheter containing the near-infrared imaging system and a working channel is placed in the sheath and advanced into the left atrium. Using a deflection mechanism in the two-lumen catheter, the catheter is positioned in close proximity to the center of the mitral valve (35). A stitching or stapling tool (33) is advanced until it is touching the joining (35) of the anterior (31) and posterior (32) leaflets when the valve closes. When the valve is closed, activation on the proximal end of the stitching tool places a single stitch at the center of the valve leaflet joining point (35).

It is appreciated that other tools could also be placed in the working channel or several working channels could be configured in the catheter. For example, stabilization of the valve leaflets would simplify the stitching procedure. In a three-lumen catheter, tools which grasp the leaflets could be employed. This would stabilize the stitching site and a stitching or stapling tool could be passed through the other channel join the valve leaflets. In fact, any methodology of joining the leaflets could be used if it existed in a catheter version.

Other valve procedures could also be performed in a similar manner, employing a near-infrared imaging catheter with one or more working channels. Introduction of a foldable annuloplasty ring is now possible since the ring and valve can be viewed and the ring can be positioned properly where it could be sutured or otherwise affixed with other tools to the valve annulus. After completion of the procedure, the ring could be viewed in detail by manipulating the catheter. In a similar manner, a prosthetic valve could be introduced, positioned and affixed in the valve orifice using specialized tools inserted in the working channel. For dilated valves, inserting a heating element through the working channel and positioning it against portions of the valve annulus and applying heat could achieve valve shrinkage. In addition, other procedures for repairing the valve could be introduced through the working channel and applied to the valve.

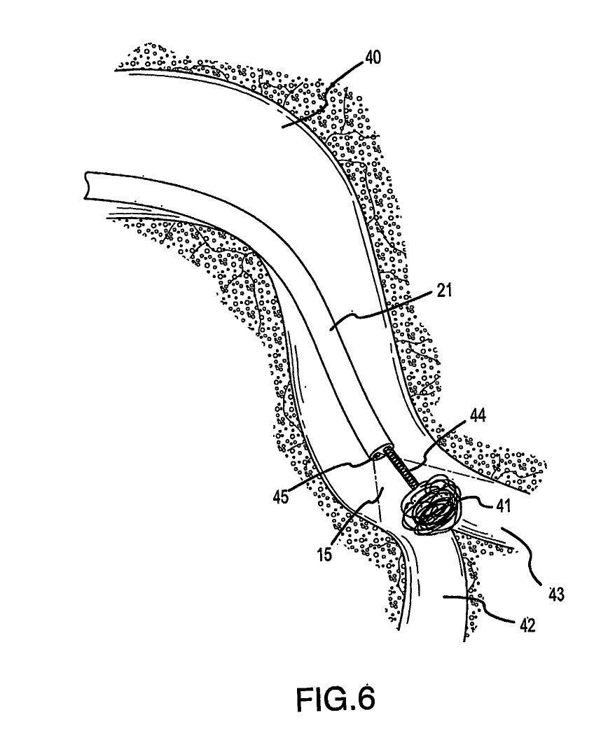

FIG. 6 is an embodiment of a catheter which views and treats thrombi in the veins and arteries. The two-lumen catheter (21) resides in a vein (40). It has one lumen (45) containing the near-infrared imaging assembly while the other lumen contains an auger mechanism (44) extended and in contact with a thrombus (41). The thrombus is lodged in a bifurcation in the vein as it splits into veins (43) and (42). The auger is rotated by a control on the proximal end of the device. As it rotates, it augers the thrombus in a proximal direction. Thrombi consist of a mixture of fibrin and red blood cells. The fibrin is “stringy” but weak. As the fibrin and red blood cells are augered into the assembly, the fibrin strands will frequently break, requiring re-application of the augering tool. Moreover, the augering changes the configuration and location of the thrombus. The near-infrared imager permits the thrombus to be in view during these changes, and provides guidance for the re-application of the augering mechanism.

Near-infrared imaging permits a wide variety of tools to be employed since there action on the thrombus is in full view of the physician. Alternative approaches include lysing with chemicals, mechanical maceration, high-pressure saline flushes and others. All of these thrombus-removing devices would benefit from viewing of the procedure. In the case of lysing, it would allow the chemical to be injected in the center of the thrombus. Mechanical maceration devices could “chip away” at the thrombus, macerating it in small sections and applying the maceration device to the remaining potion as seen on the near-infrared imager.

Reflected light images are high-resolution and can be taken at high speeds (30-100 frames/sec). Structures, which are hard, such as calcified lesions, will show little change after or during a pressure pulse since the plaque is not elastic or compressible. On the other hand, soft structures such as fibrous lesions compress and vibrate following pressure pulse application. Vulnerable plaque, which consists of a thin capsule covering a liquid lipid pool, is reported to quiver following pressure application.

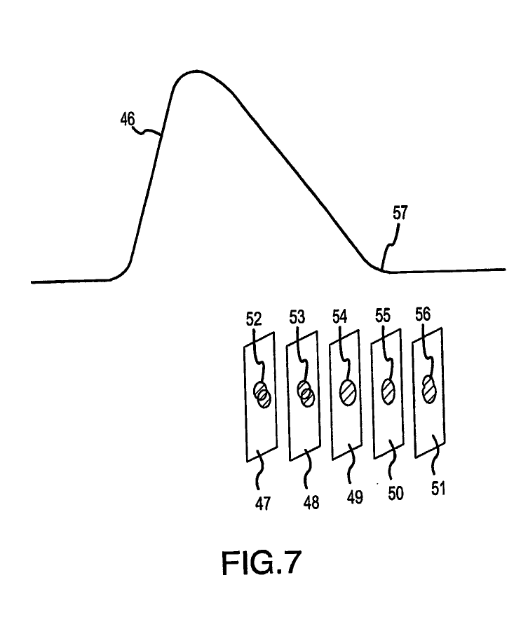

FIG. 7 is a schematic drawing of the pressure pulse (46) in an artery. The pressure pulse is about 200 milliseconds in duration. In this embodiment, a series of pictures (47-51) are taken at 30 frames/sec of a lesion during the last part of the pressure pulse and following its conclusion. The lesion pictures (52-56) are then examined to evaluate changes in confirmation. If the lesion is hard, its confirmation will not change appreciably with pressure in any of the images. If it is soft, movement and confirmation changes will occur following the pressure pulse. This procedure of evaluating conformational changes can be easily automated in an image-processing computer. The image of the lesion (52) can be stored in memory and then digitally subtracted from a picture after the pulse, say lesion image 55. Prior to the subtraction, the computer would need to line up the pictures so that the lesion was in the same place on both images. After digital subtraction, only structures moving between the images would be imaged. This image could be overlaid over the real-time image and false colored or highlighted to show areas of soft lesions. In some area of the vasculature the natural pressure pulse from the heart is too small to create conformational changes in soft tissue. An alternative is to apply a pressure pulse near the distal end of the near-infrared imaging catheter with a pressure producing transducer, such as a piezoelectric crystal.

The other application of dynamic characterization is recognizing myocardial infarcts in the ventricles. Infarcted cells do not contract; when the heart muscle cells contract the infracted cells will not change configuration. If images of portions of the ventricular surface were taken during and after contraction, those areas not moving appreciably are infracted areas.

Infrared spectroscopy has been used for decades for ascertaining the chemical composition of a sample. Most chemicals have areas of higher absorption at particular wavelengths (signature wavelength). Shining light in at the signature wavelength could image the chemical or tissue types of interest. This would create darker areas in regions where the sensed chemical is present. Using one wavelength would obscure the structure in the sensed chemical region because of the darkness produced by the sensed chemical. Also, darker regions would not necessarily be regions of the sensed chemical because many other factors could produce dark spots (i.e. insufficient illumination, poor illumination angle). If a reference wavelength were used (which did not have higher absorption for the sensed chemical or tissue type), a sensed-chemical or tissue type map could be obtained by digitally subtracting the signature wavelength from the reference wavelength. The subtracted image would contain dark spots in locations where the chemical or tissue type resides. This image can be colored or highlighted and overlaid over the reference image. This technique can be used for sensing chemical content or tissue type in any body cavity where blood is present and obscures the image.

The basic premise is as follows:

Chemicals or tissue types frequently have local absorbance peaks in the infrared regions. These are called signature wavelengths. If a structure is imaged at a wavelength corresponding to an absorbance peak, the image will be darker at the site of the chemical (it absorbs more light).

According to U.S. Pat. No. 6,178,346, if the chemical signature wavelengths (SW) is in one of following regions; Region I: 800-1400 nm, Region II: 1550 nm-1850 nm and Region III: 2100 nm-2300 nm, scattering and absorption are low enough to permit remote chemical or tissue type sensing.

If a laser diode at a signature wavelength were shined in the blood medium, followed by a laser diode pulse at a reference wavelength (RW, chosen where there is not a absorbance peak for the chemical and is in Regions I-III). If the images are digitally subtracted (RW-SW), the resultant image will contain spots where the sensed chemical or tissue type resides. This image can be highlighted or colored and added to the RW image. The resultant image consists of the RW image highlighted or colored to indicate the location of the sensed chemical or tissue type.

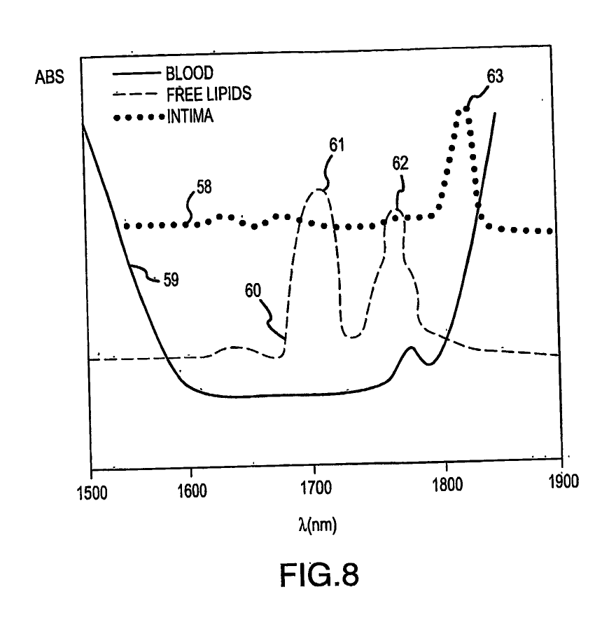

FIG. 8 is a plot of absorbance versus wavelength. In the region of low blood absorption (59) there are characteristic absorption patterns both for lipids (60) and intima tissue (58). As shown in FIG. 8, lipids have two signature wavelengths (61,62) at 1700 nm and 1760 nm. At these wavelengths, there is a local absorption peak not shared by neighboring wavelengths. Both wavelengths are in the “water window” extending from 1550-1850 nm, where near-infrared imaging is possible. An elementary approach to presenting lipid content is to make a system where two wavelengths are used sequentially: 1700 nm and the reference—1640 nm. If the image at 1640 nm is digitally subtracted from the image at 1700 nm, what remains is an indication of the lipid content of the lesion. If the digitally subtracted images were assigned highlights then an enhanced image would be possible with highlights indicating regions of high lipid content. The composite image would consist of the black and white image at 1640 nm overlaid with the lipid highlight images.

Oxidized lipids occur on the surface of advanced plaques. The main signature wavelength for oxidized lipid content occurs at 2200 nm, another water-window in infrared imaging. In the same manner as above, the image could be highlighted with another color indicating the presence of surface lipid content. This would provide highly valuable information since these tend to be Type VI lesions where the lipid pool is breaking through the surface and is indicative of imminent plaque breakage or fissure

It would be desirable to distinguish the arterial wall from plaque. Inside the arterial wall is a structure called the intima. If intima is sensed it means the arterial wall has been injured. This would be especially advantageous in atherectomy procedures where plaque is removed, without injuring the arterial wall. The most common atherectomy device, the Rotoblater, uses an electric-powered auger,which shaves tissue which enters a cavity on the side of the catheter. In fact, injury of the arterial wall has limited atherectomy to about 5% of revascularization procedures. Analysis of tissue augered out by the Rotoblator catheter demonstrates that arterial wall tissue was frequently present, indicating frequent arterial wall injury and the danger of restenosis. If the arterial wall could be highlighted or colored, the physician could titrate the atherectomy procedure, stopping when arterial intimal is sensed.

In FIG. 8, the absorption peak (63) for intima occurs at about 1830 nm. The procedure for identifying intima is as follows:

An absorption peak for arterial intima occurs at wavelength λ(1830)

A reference wavelength, 1640 nm is chosen since there is no absorption peak for arterial intima

Two laser diodes at wavelength λ(1830) and λ(1640) are fired sequentially.

Record sequentially, each wavelength image with an infrared camera.

If the images are digitally subtracted [λ(1830)−λ(1640)] the resultant image will contain spots at locations, where intima tissue is present. This image can be highlighted or colored and added to the λ(1640) image. The resultant image consists of the λ(1830) image highlighted or colored to indicate the location of arterial intima

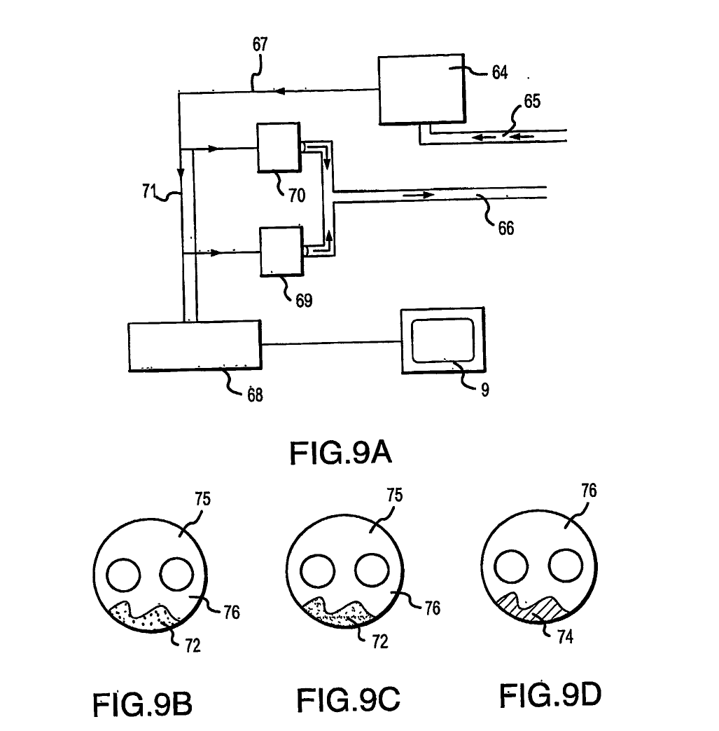

Referring to FIG. 9A, the system diagram is as follows. A computer (68) controls the firing of two lasers (69, 70). Each laser is routed by optical fibers to shine light into the illumination fibers (66) of the endoscope. Each laser is used every other picture. The reflected signal is received by the imaging fibers (65) and sent to an infrared camera (64), which sends the digital content of the picture to the computer 968) for processing. The computer performs the digital subtraction and displays the image on the monitor (9).

FIG. 9B is an image taken inside an artery (76) using the reference wavelength of 1640 nm. The image shows a bifurcation (75) and a plaque region (72). FIG. 9C is an image taken a frame later with the laser sensitive to lipids (1700 nm). Since lipids absorb this wavelength stronger than at 1640 nm, the plaque region appears darker, with the rest of the image unchanged. Digital subtraction of the images produces an image where only the plaque is present since the bifurcation is unchanged in each picture. This digital subtracted image is then superimposed and highlighted on the reference image to show regions of lipid pools in the coronary vasculature. FIG. 9D depicts the superimposed image, where the lipid-rich plaque is highlighted (74).

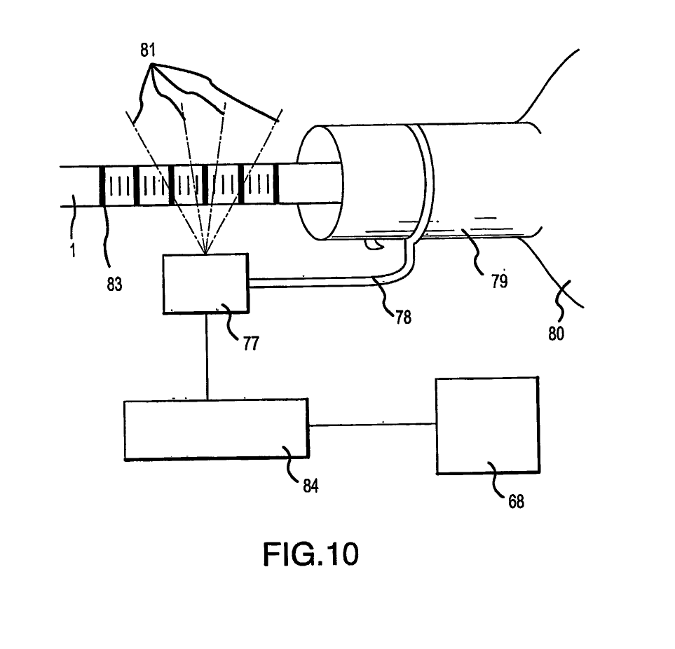

FIG. 10 shows an embodiment where images seen by the near-infrared imager can be calibrated to measure distances and object size. The distances and sizes may be measured if it is determined what the object size is at several points, and the distance between the points using a triangulation technique. This requires that the endoscope have a mechanism of determining how much it has been advanced between the points. A means of determining how far the proximal end has traveled is by measuring the travel of the distal end. FIG. 10 shows the proximal end of the near-infrared imaging catheter (1), where a reader (77) attached with a clip (78) to the catheter introducer (79) determines how much of the endoscope has advanced by reading a bar code (83) on the catheter (1). The reader signal is routed to a processor (84), which feeds the information to the near-infrared computer (68).

The measurement principle is as follows:

The angle A of the field of view is fixed and known as well as the lens aberations

If an object, identified by the on the monitor is of height H takes of half the view than it is known that arctan (H/X1)=0.5 A, corrected for lens aberrations

If the endoscope has moved to distance X2, and the object takes up one-third of the view than arctan (H/X2)=0.33 A, corrected for lens aberrations.

X2=X1+distance measured by reader

Since there are two equations with two unknowns (X1 and H), it can be solved for H the actual height of the object.

Since there are more than two images, calculation of H could be made for any two points.

Since catheters buckle when they are pushed, there is not a one-one correspondence between proximal end and distal end movement. To eliminate false readings, multiple values of H are calculated over many images, with the outliers thrown out. Thus, by determining the distance the catheter has traveled plus the size of the image, the height of the object (H) can be determined.

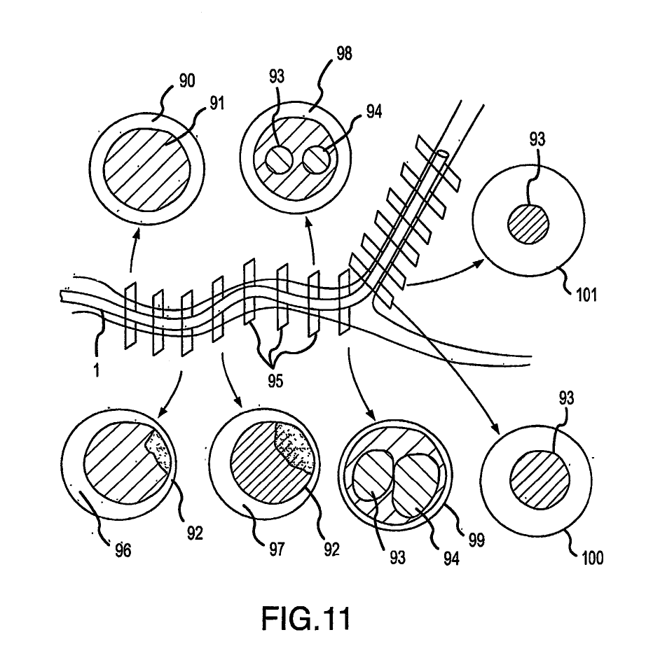

The last embodiment is a means of creating a 3-dimensional arterio-venous map of the body based images and measuring catheter positioning with a reading device described above. FIG. 11 shows the near-infrared catheter (1) entering the left branch of a bifurcation. As the catheter moves along, the reader digitally records images (95) and catheter position. The image of the first picture (90) shows a large hole (91) whose diameter can be measured by comparing it to earlier images. The third image (96) now shows a region of plaque (92) in the upper right corner. The fourth image (97) now shows the plaque (92) of bigger size. The seventh image (98) shows a distant bifurcation into two veins (93, 94) which on the eighth picture (99) become larger. The catheter passes through the upper or left bifurcation where the ninth picture (100) shows a vessel (93) of smaller diameter, which continues to decrease in size on the tenth image (101).

Recording these images along with the reader measurements permits the size of each of the objects to be determined as discussed in the previous embodiment. If stored in the computer is a sample of the vasculature passed through, corrections based on the measurements can be made to create a personal vasculature map of the patient. The image of the vasculature can be displayed in a two or three-dimensional format, similar to what is now seen in a whole-body fluoroscopy image. The interior of any part of the vasculature traversed can also be displayed showing areas of plaque formation. The second time the procedure is performed, the patient's vascular map is located in memory and the test is repeated to estimate plaque progression.

1. An intracorporeal ablation system comprising:
a near-infrared imaging catheter and an ablation catheter, wherein the imaging catheter may be positioned within 2 cm of the ablation catheter and the imaging catheter has a field of view sufficient so that a user may view the position of the ablation catheter in relation to a tissue to be ablated.

2. An intracorporeal ablation system comprising:
a catheter having at least two lumens, one of the lumens containing an optical head assembly for near-infrared imaging, the other of the lumens being capable of housing a deployable ablation component, the near-infrared imaging optical head allowing imaging within a field of view that encompasses the ablation component when it is deployed.

3. An intracorporeal ablation system comprising:
a near-infrared imaging catheter and an ablation device, the ablation device including an orientable electrode having a portion that is electrically active and another portion that is insulated, the imaging catheter providing a view of the position of the electrode, so that the insulated portion can be oriented against tissue.

4. A method of intracorporeal ablation, comprising:
inserting a near infrared imaging catheter into a patient's vasculature to a vicinity of a site to be ablated; inserting an ablation device into the vicinity of a site to be ablated; positioning the ablation device to a desired site using the imaging catheter; and ablating the tissue with the ablating device.

5. The method of claim 4, wherein the ablation device is positioned so that it is pressed against tissue to be ablated.

6. An intracorporeal suturing device, comprising:
an infrared-imaging catheter having at least two lumens, one of the lumens including an optical head assembly to provide for imaging, and the other lumen housing a stitching or stapling tool, the optical head assembly providing for imaging of the stitching or stapling tool when the tool is deployed.

7. A method of intracorporeal dynamic characterization, comprising the steps of introducing an infrared imaging catheter into a body to be imaged, taking a series of images within the body during an event such as a pressure pulse, digitally subtracting some of the series of images from some other of said images, and evaluating the result of the digital subtraction to identify movement and conformational changes.

8. The method of claim 7, further including the step of introducing a pressure pulse within the body, such as with a piezoelectric crystal.

9. A method of identifying the presence of a chemical composition at an intracorporeal site, comprising the steps of:
imaging the site with a near infrared imaging catheter as a reference wavelength, to form a first image;
imaging the site with the catheter at a signature wavelength, the signature being selected to corresponded to a local absorption peak of the chemical composition in the infrared regions and being selected to be different from the reference wavelength, to form a second image; and
subtracting the images to identify the chemical composition.

10. A method of determining the size of an object intracorporeally, comprising:
taking an image of an intracorporeal object using an infrared imaging catheter;
advancing the imaging catheter a known distance;
taking another image of the object;
determining the size of the object by comparing the relative sizes of the object in the images and computing the size using the known distance that the catheter was advanced.

11. A method of creating an arterio-venous map, comprising the steps of introducing an infrared-imaging catheter into a body, taking an image with the catheter at a first position, moving the catheter, taking another image, repeating the preceding two steps multiple times to create a series of images within a body taken at multiple locations, and using the series of images to form an image of the body's vasculature.

12. The method of claim 11, comprising creating a second image at another time than the first image, and comparing the first and second images to determine a change in the body such as the formation of plaque.
